(19) United States
(12) Patent Application Publication
(10) Pub. No.: US 2022/0304352 A1
(43) Pub. Date: Sep. 29, 2022

(54) TASTE-MODIFYING COMPOUNDS AND USES THEREOF

(71) Applicant: Firmenich SA, Satigny (CH)

(72) Inventors: Xian-Wen GAN, Shanghai (CN); Eric FREROT, Satigny (CH)

(73) Assignee: Firmenich SA, Satigny (CH)

(21) Appl. No.: 17/616,847

(22) PCT Filed: Jun. 26, 2020
(86) PCT No.: PCT/EP2020/068096

§ 371 Date: Dec. 6, 2021

(30) Foreign Application Priority Data
Aug. 15, 2019 (CN) PCT/CN2019/100810
Oct. 22, 2019 (EP) 19204703.3

(51) Int. Cl. A23L 27/00; A23L 27/21

(52) U.S. Cl. CPC A23L 27/88; A23L 27/21

(57) ABSTRACT

The present disclosure generally relates to compounds useful as taste modifiers, particularly as compounds useful for enhancing umami taste, and their use in various comestible products, such as food and beverage products.

### CROSS-REFERENCE TO RELATED APPLICATIONS

This present application claims priority to PCT App. No. PCT/CN2019/100810, filed Aug. 15, 2019, and to European Patent App. No. 19204703.3, filed Oct. 22, 2019, both of which are hereby incorporated by reference as though set forth herein in their entireties.

### TECHNICAL FIELD

The present disclosure generally relates to compounds useful as taste modifiers, particularly as compounds useful for enhancing umami taste, and their use in various comestible products, such as food and beverage products.

### DESCRIPTION OF RELATED ART

Flavor modifiers are substances added to supplement, enhance, or modify the original flavor of a flavored article. Flavor is defined as the combined perception of taste, smell or aroma and chemical feeling factors. The perception of flavor is a result of the chemical stimulation of receptors in both the oral and nasal cavities. The basic tastes are sweet, sour, salty and bitter. Umami, described as another basic taste, enhances the taste effect of other ingredients and components of the flavor profile. These basic tastes, including umami and certain trigeminal effects are perceived in the buccal cavity. Aroma may be the smell emanating from food before it is consumed or the flavor perceived while chewing and swallowing a product.

Flavor and/or aroma modifiers may be added to foods (including beverages), personal or household care products, pharmaceutical preparations, or other compositions to increase acceptance of products by enhancing desirable flavors and/or aromas or by masking or eliminating undesirable attributes. Flavor modifiers may be used to alter the taste and/or aroma of ingestible foods, nutraceuticals and pharmaceuticals, as well as oral and personal care products (e.g., mouthwash, toothpaste, cosmetics, perfumes and the like), or products that may be found in and around homes, businesses, and the like.

It has long been a goal to improve the quality of food products and to provide new and different flavor and aroma sensations to such products. Commercial production of foods expected to have a relatively long shelf-life often requires the use of processing conditions, storage conditions and/or addition of ingredients that may produce undesirable off-tastes in the food compositions. Typical solutions to taste problems are ineffective often due to the high cost of ingredients and manufacturing. The use of flavor modifiers could eliminate or substantially reduce the undesirable off-tastes in food compositions as well as improve the overall taste perception of the food and/or provide new and novel taste experiences. Accordingly, it would be desirable to supply a flavor modifier that could provide such flavor improvements and novelty to foods and preferably contribute these benefits in a cost effective manner.

### SUMMARY

In a first aspect, the disclosure provides taste-modifying compounds, which are compounds of formula (I) or salts thereof:

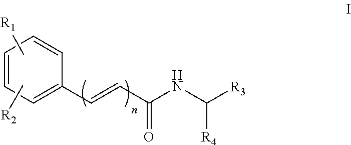

wherein

n is an integer ranging from 0 to 2;

R₁ and R₂, are independently a hydrogen atom, C₁₋₅ alkyl, —O—(C₁₋₅ alkyl), or —OH;

or R₁ and R₂ may optionally combine when attached to adjacent carbon atoms to form an —OCH₂O— group;

R₃ is —COOH or —CH₂OH; and

R₄ is an acyclic saturated or unsaturated hydrocarbon, or an acyclic saturated or unsaturated hydrocarbon substituted by heteroatoms, preferably O, N, or S atoms, or a carbocyclic or heterocyclic group;

wherein double bond is in a conformation E or Z.

In some embodiments, the compound is not hippuric acid, or a compound with CAS number 1643580-85-1, 851669-61-9, 851669-64-2, 851668-86-5, 851668-89-8, and 851668-91-2.

In a second aspect, the disclosure provides taste-modifying compounds, which are compounds of formula Ia or salts thereof:

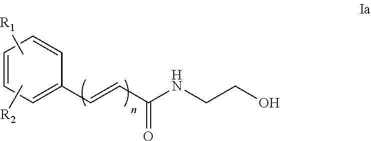

wherein:

n is an integer from 0 to 2; and

R₁ and R₂, are independently a hydrogen atom, C₁₋₅ alkyl, —O—(C₁₋₅ alkyl), or —OH;

or R₁ and R₂ may optionally combine when attached to adjacent carbon atoms to form an —OCH₂O— group.

In a third aspect, the disclosure provides a taste-modifying compound, which is a compound of formula II or a salt thereof:

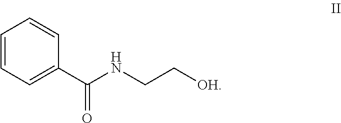 [SMILES: O=C(NCCO)c1ccccc1]

In a fourth aspect, the disclosure provides a taste-modifying compound, which is a compound of formula III or a salt thereof:

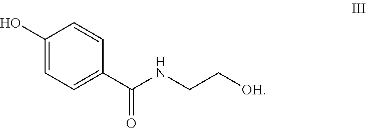 [SMILES: O=C(NCCO)c1ccc(O)cc1]

In a fifth aspect, the disclosure provides a taste-modifying compound, which is a compound of formula IV or a salt thereof:

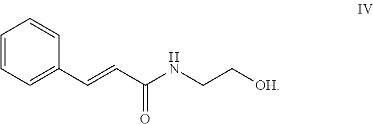 [SMILES: O=C(/C=C/c1ccccc1)NCCO]

In a sixth aspect, the disclosure provides a taste-modifying compound, which is a compound of formula V or a salt thereof:

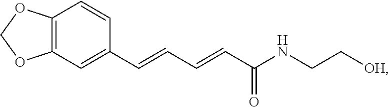 [SMILES: O=C(/C=C/C=C/c1ccc2c(c1)OCO2)NCCO]

In a seventh aspect, the disclosure provides a taste-modifying compound, which is a compound of formula Ib or a salt thereof:

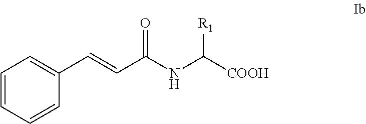

wherein:

R₁ is a side chain of an amino acid, selected from the group consisting of alanine (Ala), aspartic acid (Asp), phenylalanine (Phe), glutamic acid (Glu), isoleucine (Ile), lysine (Lys), leucine (Leu), asparagines (Asn), glutamine (Gln), arginine (Arg), valine (Val), glycine (Gly), cysteine (Cys), theanine, histidine (His), methionine (Met), serine (Ser), threonine (Thr), tryptophan (Trp), and tyrosine (Tyr).

In an eighth aspect, the disclosure provides a taste-modifying compound, which is a compound of formula VI or a salt thereof:

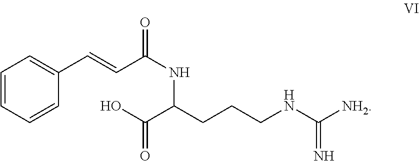 [SMILES: N=C(N)NCCCC(NC(=O)/C=C/c1ccccc1)C(=O)O]

In a ninth aspect, the disclosure provides a taste-modifying compound, which is a compound of formula VII or a salt thereof:

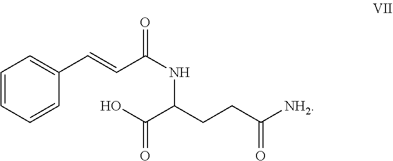 [SMILES: NC(=O)CCC(NC(=O)/C=C/c1ccccc1)C(=O)O]

In a tenth aspect, the disclosure provides a taste-modifying compound, which is a compound of formula XVI or a salt thereof:

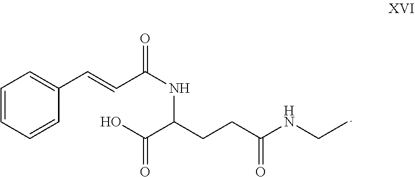 [SMILES: CCNC(=O)CCC(NC(=O)/C=C/c1ccccc1)C(=O)O]

In an eleventh aspect, the disclosure provides uses of a compound of any of the first through the tenth aspects to enhance an umami taste of a flavored article. In some embodiments, the flavored article comprises an umami ingredient.

In a twelfth aspect, the disclosure provides uses of a compound of any of the first through the tenth aspects to enhance a salty taste of a flavored article. In some embodiments, the flavored article comprises a salty ingredient.

In a thirteenth aspect, the disclosure provides methods of enhancing an umami taste of a flavored article, comprising introducing to a flavored article a compound of any of the first through the tenth aspects. In some embodiments, the flavored article comprises an umami ingredient.

In a fourteenth aspect, the disclosure provides methods of enhancing a salty taste of a flavored article, comprising introducing to a flavored article a compound of any of the first through the tenth aspects. In some embodiments, the flavored article comprises a salty ingredient.

In a fifteenth aspect, the disclosure provides a flavored article comprising a compound of any of the first through the tenth aspects. In some embodiments, the flavored article comprises a salty ingredient, an umami ingredient, or a combination thereof. In some embodiments, the flavored article is a food product or a beverage product.

### BRIEF DESCRIPTION OF DRAWINGS

FIG. 1 is a bar graph showing that Compound II at a dosage of 5 ppm enhanced saltiness and umami in a 0.25% NaCl base and a 0.05% MSG base solutions in subjects with and without a nose-clip.

FIG. 2 is a bar graph showing that Compound III at a dosage of 5 ppm enhanced saltiness and umami in a 0.25% NaCl base and a 0.05% MSG base solutions in subjects with and without a nose-clip.

FIG. 3 is a bar graph showing that Compound IV at a dosage of 10 ppm enhanced saltiness and umami in a 0.25% NaCl base and a 0.05% MSG base solutions in subjects with and without a nose-clip.

FIG. 4 is a bar graph showing that Compound V at a dosage of 20 ppm enhanced umami in a 0.05% MSG base and masked KCl taste in a 0.25% KCl base solutions in subjects with and without a nose-clip.

FIGS. 5A and 5B show bar graphs showing that Compound VI at a dosage of 10 ppm enhanced saltiness (FIG. 5A) and umami (FIG. 5B) in a 0.25% NaCl base and a 0.05% MSG base solutions in subjects with and without a nose-clip.

### DETAILED DESCRIPTION

In one aspect, the disclosure provides taste-modifying compounds, which are compounds of formula I or salts thereof:

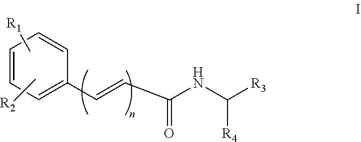

wherein

n is an integer ranging from 0 to 2;

R₁ and R₂, are independently a hydrogen atom, C₁₋₅ alkyl, —O—(C₁₋₅ alkyl), or —OH;

or R₁ and R₂ may optionally combine when attached to adjacent carbon atoms to form an —OCH₂O— group;

R₃ is —COOH or —CH₂OH; and

R₄ is an acyclic saturated or unsaturated hydrocarbon, or an acyclic saturated or unsaturated hydrocarbon substituted by heteroatoms, preferably O, N, or S atoms, or a carbocyclic or heterocyclic group;

wherein double bond is in a conformation E or Z.

In some embodiments, the compound is not hippuric acid, or a compound with CAS number 1643580-85-1, 851669-61-9, 851669-64-2, 851668-86-5, 851668-89-8, and 851668-91-2.

In a related aspect, the disclosure provides taste-modifying compounds, which are compounds of formula Ia or salts thereof:

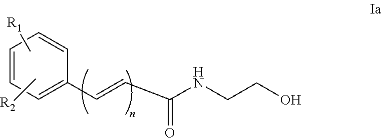

wherein:

n is an integer from 0 to 2; and

R₁ and R₂, are independently a hydrogen atom, C₁₋₅ alkyl, —O—(C₁₋₅ alkyl), or —OH;

or R₁ and R₂ may optionally combine when attached to adjacent carbon atoms to form an —OCH₂O— group.

Taste-modifying compounds of the present disclosure also include the following compounds:

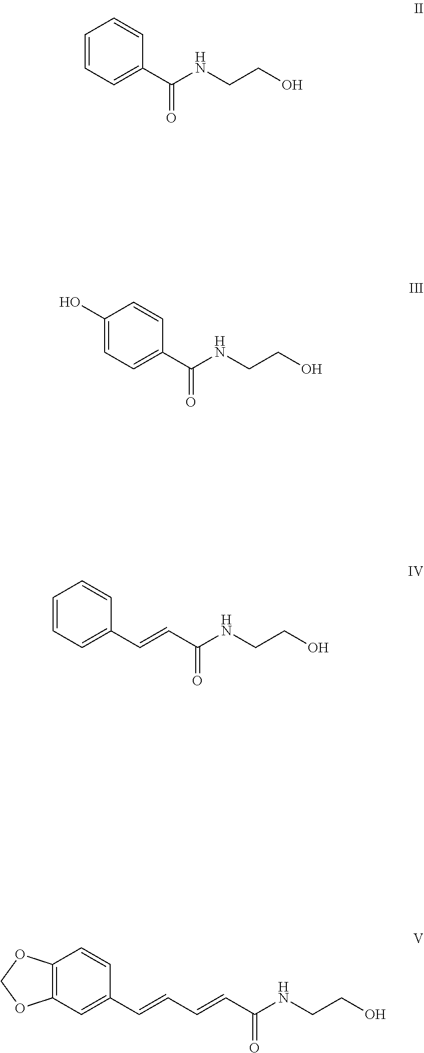 [SMILES: O=C(NCCO)c1ccccc1 ; O=C(NCCO)c1ccc(O)cc1 ; O=C(/C=C/c1ccccc1)NCCO ; O=C(/C=C/C=C/c1ccc2c(c1)OCO2)NCCO]

In another aspect, the disclosure provides a taste-modifying compound, which is a compound of formula Ib or a salt thereof:

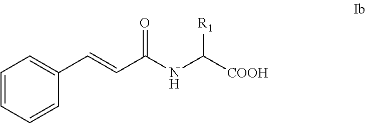

wherein:

R₁ is a side chain of an amino acid, selected from the group consisting of alanine (Ala), aspartic acid (Asp), phenylalanine (Phe), glutamic acid (Glu), isoleucine (Ile), lysine (Lys), leucine (Leu), asparagines (Asn), glutamine (Gln), arginine (Arg), valine (Val), glycine (Gly), cysteine (Cys), theanine, histidine (His), methionine (Met), serine (Ser), threonine (Thr), tryptophan (Trp), and tyrosine (Tyr).

Taste-modifying compounds of the present disclosure also include the following compounds:

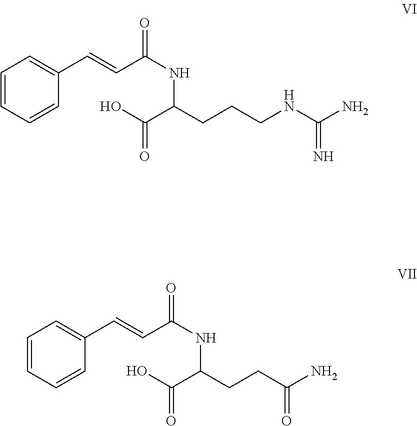 [SMILES: N=C(N)NCCCC(NC(=O)/C=C/c1ccccc1)C(=O)O ; NC(=O)CCC(NC(=O)/C=C/c1ccccc1)C(=O)O]

The taste-modifying compounds can be included in flavored articles at any suitable concentrations. In some embodiments, the taste-modifying compounds of any of the foregoing aspects or embodiments thereof are present in the flavored article at a concentration ranging from 0.1 ppm to 1000 ppm, or from 5 ppm to 20 ppm.

A “salty ingredient” as used herein includes salts known in the art for use in a flavored article. Non-limiting examples of a salty ingredient include sodium chloride, potassium chloride, magnesium chloride, calcium chloride, and similar lithium, potassium, ammonium or other alkali earth salts.

An “umami ingredient” as used herein includes umami ingredients know in the art for use in a flavored article. Non-limiting examples of an umami ingredient include monosodium glutamate (MSG), ribotides, 5336 (FEMA4233) (Senomyx), 5807 (FEMA4232) (Senomyx), Ericamide (FEMA4877) (Firmenich), Rubemamine (FEMA4310), Rubescenamine (FEMA4773) and Geranylamine cyclopropane carboxylate (FEMA4267).

In some aspects, a flavored article is provided comprising a compound according to the present invention.

As used herein, the term “flavored article” includes, but is not limited to foodstuffs of all kinds, confectionery products, baked products, sweet products, savory products, fermented products, dairy products, beverages, oral care products, nutraceuticals and pharmaceuticals.

Exemplary foodstuffs include, but are not limited to, chilled snacks, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, meat analogues, frozen pizza, chilled pizza, soup, canned soup, dehydrated soup, instant soup, chilled soup, UHT soup, frozen soup, pasta, canned pasta, dried pasta, chilled/fresh pasta, noodles, plain noodles, instant noodles, cups/bowl instant noodles, pouch instant noodles, chilled noodles, snack noodles, dried food, dessert mixes, sauces, dressings and condiments, herbs and spices, spreads, jams and preserves, honey, chocolate spreads, nut-based spreads, and yeast-based spreads.

In some aspects, the concentration of a compound of the present invention, when added to the flavored article is, for example, about 0.0001, 0.0005, 0.001, 0.002, or 0.003, or, 0.004, or 0.005, or 0.006, or 0.007, or 0.008, or 0.009, or 0.01, or 0.02, or 0.03, or, 0.04, or 0.05, or 0.06, or 0.07, or 0.08, or 0.09, or 0.1, or 0.15, or 0.2 weight percent, based on the total weight of the flavored article.

In some embodiments, the composition according to an aspect presented herein may be employed in either liquid form and/or dried form. When employed in the latter form, suitable drying means such as spray drying the oil may be used. Alternatively, the composition may be absorbed onto water soluble materials, such as cellulose, starch, sugar, maltodextrin, gum arabic and so forth or may be encapsulated. The techniques for preparing such dried forms are well-known in the art.

In some aspects, the composition according to an aspect presented herein may be used in distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

In one aspect, the composition according to an aspect presented herein may be in a crystalline, granulated, or powder form.

Exemplary confectionery products include, but are not limited to, chewing gum (which includes sugarized gum, sugar-free gum, functional gum and bubble gum), centerfill confections, chocolate and other chocolate confectionery, medicated confectionery, lozenges, tablets, pastilles, mints, standard mints, power mints, chewy candies, hard candies, boiled candies, breath and other oral care films or strips, candy canes, lollipops, gummies, jellies, fudge, caramel, hard and soft panned goods, toffee, taffy, liquorice, gelatin candies, gum drops, jelly beans, nougats, fondants, combinations of one or more of the above, and edible flavor compositions incorporating one or more of the above.

Exemplary baked products include, but are not limited to, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes.

Exemplary sweet products include, but are not limited to, breakfast cereals, ready-to-eat (“rte”) cereals, family breakfast cereals, flakes, muesli, other ready to eat cereals, children's breakfast cereals, hot cereals.

Exemplary savory products include, but are not limited to, salty snacks (potato chips, crisps, nuts, tortilla-tostada, pretzels, cheese snacks, corn snacks, potato-snacks, ready-to-eat popcorn, microwaveable popcorn, pork rinds, nuts, crackers, cracker snacks, breakfast cereals, meats, aspic, cured meats (ham, bacon), luncheon/breakfast meats (hotdogs, cold cuts, sausage), tomato products, margarine, peanut butter, soup (clear, canned, cream, instant, ultrahigh temperature “UHT”), canned vegetables, and sauces.

Exemplary dairy products include, but are not limited to, cheese, cheese sauces, cheese-based products, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts.

Exemplary beverages include, but are not limited to, flavored water, soft drinks, fruit drinks, coffee-based drinks, tea-based drinks, juice-based drinks (includes fruit and vegetable), milk-based drinks, gel drinks, carbonated or non-carbonated drinks, powdered drinks, alcoholic or non-alcoholic drinks, and ready to drink liquid formulations of these beverages.

Exemplary fermented foods include, but are not limited to, cheese and cheese products, meat and meat products, soy and soy products, fish and fish products, grain and grain products, fruit and fruit products.

### EXAMPLES

The present disclosure is illustrated by, but not limited to, the following examples.

### Example 1: Synthesis of Compound (II)

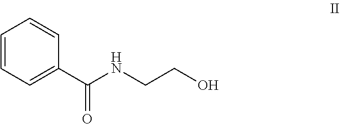 [SMILES: O=C(NCCO)c1ccccc1]

Benzoyl chloride (1.00 g, 7.1 mmol) was added to a mixture of ethanolamine (0.48 g, 7.8 mmol) and triethylamine (1.43 g, 14.1 mmol) in 20 mL of DCM. After stirring for 3 h at room temperature (rt), the mixture was added 50 mL water, then extracted with DCM. The aqueous phase was dried under reduced pressure. The concentrated crude product was then purified by ODS to afford white solid (0.23 g, 19.2%).

### Example 2: Synthesis of Compound (III)

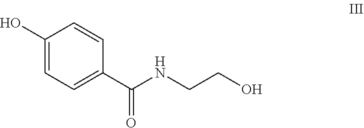 [SMILES: O=C(NCCO)c1ccc(O)cc1]

Paraben (4.00 g, 26.3 mmol) and ethanolamine (1.61 g, 26.3 mmol) was added in a round bottom flask with a Dean-Stark. Resulting mixture was stirred for 2.5 hour at 160° C. The ethanolamine was removed under reduced pressure, and then purified by silica gel to afford white solid (0.27 g, 8.2%).

### Example 3: Synthesis of Compound (IV)

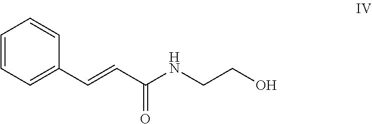 [SMILES: O=C(/C=C/c1ccccc1)NCCO]

Cinnamoyl chloride (1.00 g, 6.0 mmol) was added to a mixture of ethanolamine (0.40 g, 6.6 mmol) and trimethylamine (1.22 g, 12.0 mmol) in 25 mL of DCM. After stirring for 3 h at rt, the mixture was added 50 mL water, then extracted with DCM. The organic phase was washed with brine and dried over Na₂SO₄. The concentrated crude product was then purified by silica gel to afford white solid (0.60 g, 52.3%).

### Example 4: Synthesis of Compound (V)

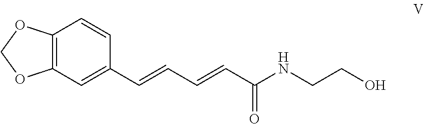 [SMILES: O=C(/C=C/C=C/c1ccc2c(c1)OCO2)NCCO]

(E,E)-Piperic acid chloride (0.50 g, 2.1 mmol) was diluted in 10 ml of dichloromethane and triethylamine (0.59 mL, 4.2 mmol). The ethanolamine (0.13 g, 2.1 mmol) was added and the reaction was stirred overnight. The reaction was successively washed with 5% KHSO₄, 5% NaHCO₃ and brine. The amide was recrystallized from EtOAc. Yields 43% were obtained.

### Example 5: Sensory Tests

A panel consisting of 25 trained panelists evaluated samples at 5, 10 or 20 ppm for taste properties (salt and/or umami, or KCl taste) on a scale of −5 to 5 (−5 denoted strong masking effect and 5 denoted strong enhancing effect, 0 being the intensity of a reference water solution containing 0.25% NaCl or 0.25% KCl or 0.05% MSG or 1.5% chicken broth base).

Chicken broth base:

| Ingredient | Dosage (%) |
|---|---|
| NaCl | 0.36 |
| KCl | 0.18 |
| MSG | 0.315 |
| Ribotide | 0.015 |
| Sucrose | 0.069 |
| Chicken fat | 0.03 |
| Garlic powder | 0.003 |
| Onion powder | 0.009 |
| Yeast | 0.042 |
| Chichen | 0.0075 |
| powder | |
| Pepper white | 0.0015 |
| Maltrodextrin | 0.3255 |
| Corn starch | 0.12 |
| Malic acid | 0.0045 |
| Curry powder | 0.0015 |
| Flavor 1 | 0.0045 |
| Flavor 2 | 0.003 |
| Flavor 3 | 0.009 |

### 1) Sensory Evaluation

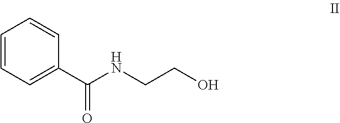 [SMILES: O=C(NCCO)c1ccccc1]

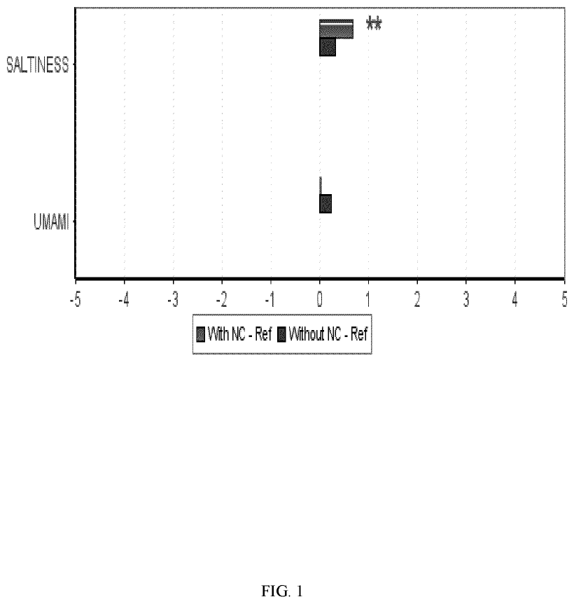

Compound II at a dosage of 5 ppm enhanced umami without nose-clip in 0.05% MSG base, enhanced saltiness without nose-clip, and significantly enhanced saltiness with nose-clip in 0.25% NaCl base (at 99% of confidence level). FIG. 1.

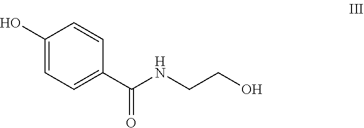 [SMILES: O=C(NCCO)c1ccc(O)cc1]

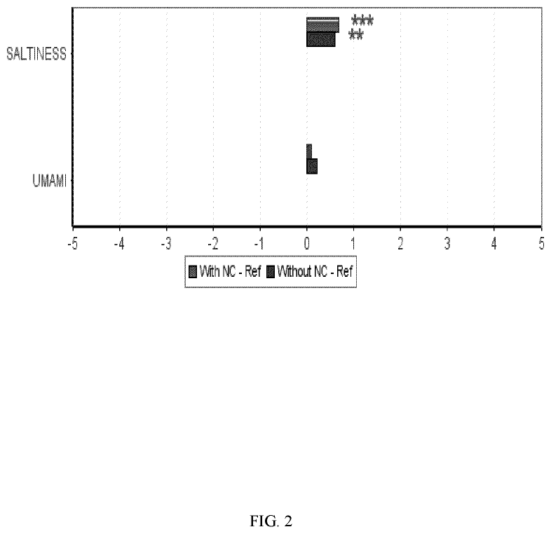

Compound III at a dosage of 5 ppm enhanced umami with and without nose-clip in 0.05% MSG base, and significantly enhanced saltiness with and without nose-clip in 0.25% NaCl base (at least 99% of confidence level). FIG. 2.

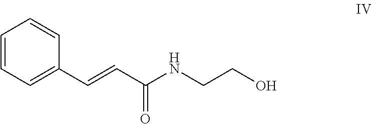 [SMILES: O=C(/C=C/c1ccccc1)NCCO]

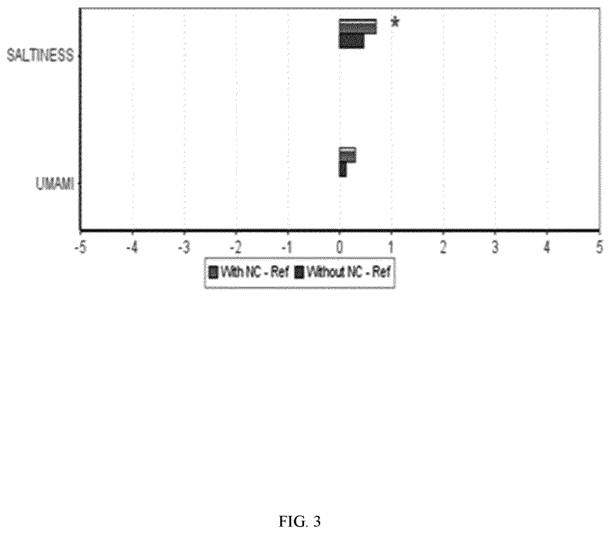

Compound IV at a dosage of 10 ppm enhanced umami with and without a nose-clip in 0.05% MSG base, enhanced saltiness without nose-clip and significantly enhanced saltiness with nose-clip in 0.25% NaCl base (at 95% of confidence level). FIG. 3.

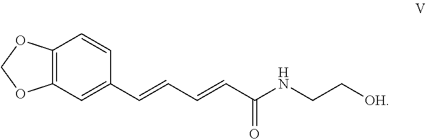 [SMILES: O=C(/C=C/C=C/c1ccc2c(c1)OCO2)NCCO]

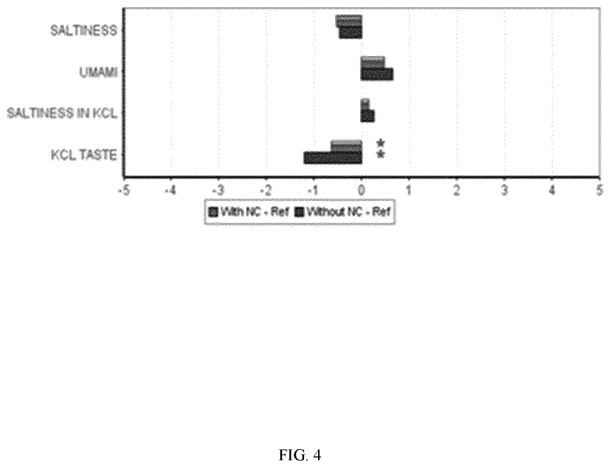

Compound V at a dosage of 20 ppm enhanced umami with and without nose-clip in 0.05% MSG base, enhanced saltiness and significantly masked KCl taste with and without nose-clip in 0.25% KCl base (at 95% of confidence level). FIG. 4.

### 2) Application Test

| | | | Without | Significant |
|---|---|---|---|---|
| Matrix | Taste | Compound | nose-clip | level |
| 1.5% chicken | Salt | II | 0.61 | *** |
| broth base | | | | |
| 1.5% chicken | Umami | II | 0.52 | * |
| broth base | | | | |
| 1.5% chicken | Salt | III | 0.4 | * |
| broth base | | | | |
| 1.5% chicken | Umami | III | 0.51 | * |
| broth base | | | | |

Samples II and III at 5 ppm had strong both saltiness and umami taste enhancing effects in chicken broths. These results were significant at least 95% confidence level (two-tail paired student test).

### Example 6: Synthesis of Compounds VI Through XVI

Cinnamoyl chloride (11 mmol) was added in three portions to a round bottom flask containing a mixture of an amine acid component (see below) (10 mmol) and sodium hydroxide (40 mmol) in 100 mL acetone/water solution (1:1) (w/w). Resulting mixture was stirred for 2 hour at room temperature and the mixture was then added diluted HCl aq. solution (1 mol/L) to adjust pH to 2-3. The acetone was removed under vacuum and extracted with Et₂O. The aqueous phase was dried, purified by ODS or silica gel or crystallization to afford final product.

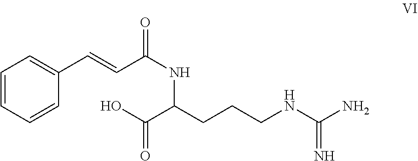 [SMILES: N=C(N)NCCCC(NC(=O)/C=C/c1ccccc1)C(=O)O]

Arginine as starting material. Yield: 8.3%.

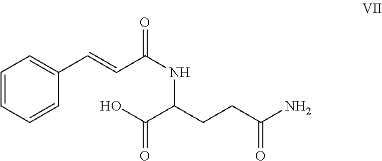 [SMILES: NC(=O)CCC(NC(=O)/C=C/c1ccccc1)C(=O)O]

Glutamine as starting material. Yield: 15.3%.

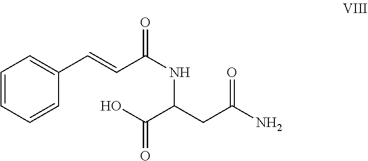 [SMILES: NC(=O)CC(NC(=O)/C=C/c1ccccc1)C(=O)O]

Asparagine as starting material. Yield: 30.9%.

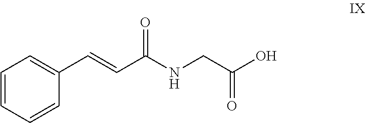 [SMILES: O=C(O)CNC(=O)/C=C/c1ccccc1]

Glycine as starting material. Yield: 19.7%.

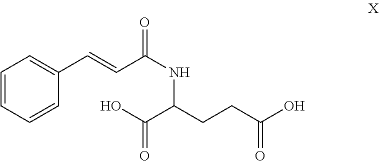 [SMILES: O=C(O)CCC(NC(=O)/C=C/c1ccccc1)C(=O)O]

Glutamic acid as starting material. Yield: 34.2%.

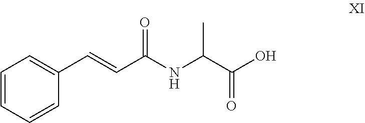 [SMILES: CC(NC(=O)/C=C/c1ccccc1)C(=O)O]

Alanine as starting material. Yield: 29.8%.

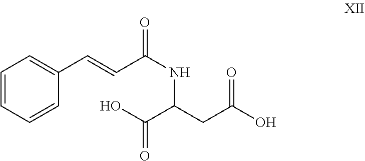 [SMILES: O=C(O)CC(NC(=O)/C=C/c1ccccc1)C(=O)O]

Asparatic acid as starting material. Yield: 26.0%.

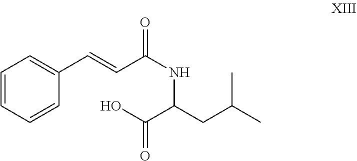 [SMILES: CC(C)CC(NC(=O)/C=C/c1ccccc1)C(=O)O]

Leucine as starting material. Yield: 32.0%.

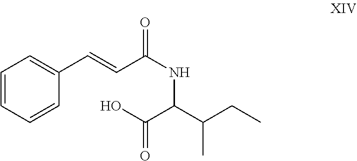 [SMILES: CCC(C)C(NC(=O)/C=C/c1ccccc1)C(=O)O]

Isoleucine as starting material. Yield: 17.0%.

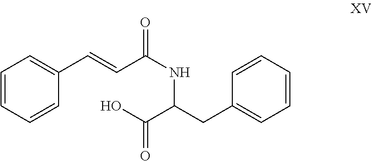 [SMILES: O=C(/C=C/c1ccccc1)NC(Cc1ccccc1)C(=O)O]

Phenylalanine as starting material. Yield: 25.8%.

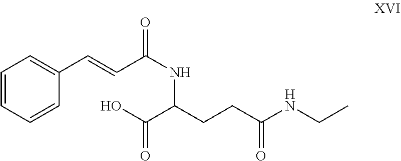 [SMILES: CCNC(=O)CCC(NC(=O)/C=C/c1ccccc1)C(=O)O]

Theanine as starting material. Yield: 16.5%.

### Example 7: Sensory Tests

A panel consisted of 25 trained panelists evaluated samples at 5 ppm for taste properties (salt and/or umami) on a scale of −5 to 5 (−5 denoted strong masking effect and 5 denoted strong enhancing effect, 0 being the intensity of a reference water solution containing 0.25% NaCl or 0.05% MSG or 1.5% chicken broth base).
1) Sensory Evaluation

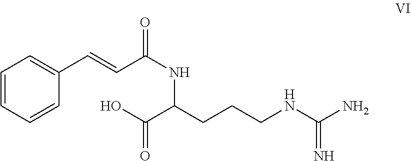 [SMILES: N=C(N)NCCCC(NC(=O)/C=C/c1ccccc1)C(=O)O]

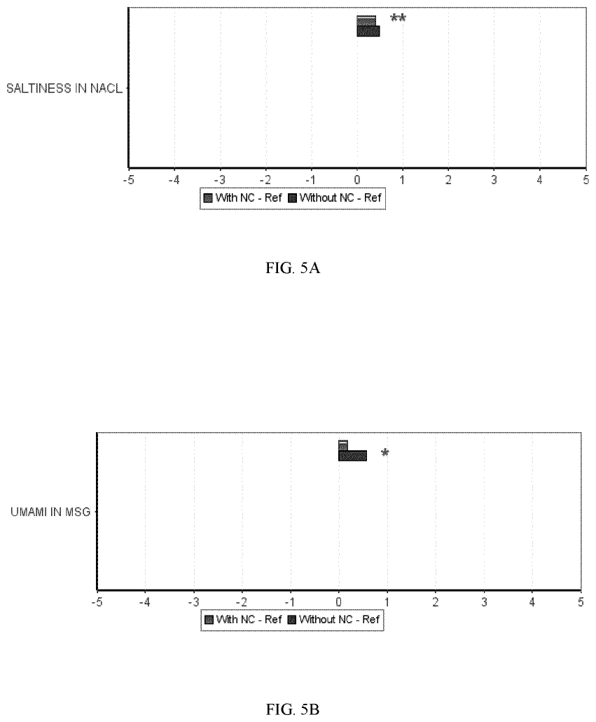

Compound VI at a dosage of 5 ppm significantly enhanced saltiness intensity in 0.25% NaCl base at a 99% of confidence level with nose-clip. Compound VI also significantly enhanced umami intensity in 0.05% MSG base at a 95% of confidence level without nose-clip. FIG. 5.
2) Application Test

| | | Without | |
|---|---|---|---|
| Matrix | Taste | nose-clip | Significant level |
| 1.5% chicken broth base | Salt | 0.49 | ** |
| 1.5% chicken broth base | Umami | 0.66 | ** |

Compound VI at a dosage of 5 ppm, had strong saltiness and umami taste enhancing effects in chicken broth. These effects are significant at confidence level of 99% (two-tail paired student test).

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1-8. (canceled)

9. A method of enhancing an umami or salty taste of a flavored article, the method comprising introducing a taste-modifying compound to the flavored article, wherein the taste-modifying compound is a compound of formula Ib or a salt thereof: 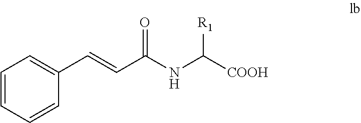
wherein:
R₁ is a side chain of an amino acid, selected from the group consisting of alanine (Ala), aspartic acid (Asp), phenylalanine (Phe), glutamic acid (Glu), isoleucine (Ile), lysine (Lys), leucine (Leu), asparagines (Asn), glutamine (Gln), arginine (Arg), valine (Val), glycine (Gly), cysteine (Cys), theanine, histidine (His), methionine (Met), serine (Ser), threonine (Thr), tryptophan (Trp), and tyrosine (Tyr).

10. The method of claim 9, wherein the taste-modifying compound is a compound of formula VI or a salt thereof:

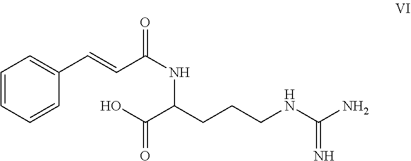 [SMILES: N=C(N)NCCCC(NC(=O)/C=C/c1ccccc1)C(=O)O]

11. The method of claim 9, wherein the taste-modifying compound is a compound of formula VII or a salt thereof:

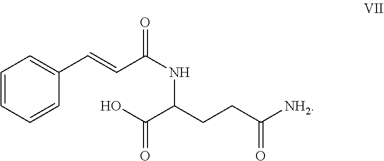 [SMILES: NC(=O)CCC(NC(=O)/C=C/c1ccccc1)C(=O)O]

12. The method of claim 9, wherein the taste-modifying compound is a compound of formula XVI or a salt thereof:

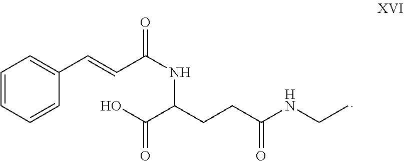 [SMILES: CCNC(=O)CCC(NC(=O)/C=C/c1ccccc1)C(=O)O]

13. The method of claim 9, wherein the flavored article comprises a salty ingredient, an umami ingredient, or a combination thereof.
